(19) United States
(12) Patent Application Publication
(10) Pub. No.: US 2007/0110738 A1
(43) Pub. Date: May 17, 2007

(54) Receptor antagonists as therapeutic agents for iNOS generating illness

(75) Inventors: Robert J. Webber, Las Vegas, NV (US); Douglas S. Webber, Los Angeles, CA (US)

Correspondence Address: THEODORE J. BIELEN JR., 1390 WILLOW PASS ROAD, SUITE 1020, CONCORD, CA, 94520, US

(73) Assignee: DSX THERAPEUTICS, LLC

(21) Appl. No.: 11/280,049

(22) Filed: Nov. 16, 2005

(51) Int. Cl. A61K 38/44

(52) U.S. Cl. 424/94.4

(57) ABSTRACT

A treatment for SIRS, sepsis, severe sepsis and septic shock in a mammalian subject by utilizing a method and therapeutic agent for inhibiting the binding of particulate iNOS to cells. The agent takes the form of a cellular receptor antagonist.

### BACKGROUND OF THE INVENTION

The present invention relates to novel and useful therapeutic agents for the treatment for systemic inflammatory response syndrome (SIRS) which is also called pre-sepsis, sepsis, severe sepsis, and septic shock based upon inhibition of the binding to cellular receptors by antagonists of particulate inducible nitric oxide synthase (iNOS).

Nitric oxide synthase (NOS) is an enzyme which is found in humans and other mammals. Three isoforms of NOS have been identified. In the body nNOS and eNOS are constitutively expressed in the cells in which they are found. However, iNOS is not constitutively expressed, but is known to be induced by a number of cytokines, lipopolysaccarides (LPS), and other mediators of the inflammatory response. Specifically, iNOS has been associated with certain pathologies. Notably, iNOS in the blood heralds the onset of sepsis, severe sepsis, and septic shock in humans. Sepsis is estimated to kill more than 250,000 people annually in the United States alone. Of the people who develop sepsis, approximately thirty percent die from this life threatening pathophysiology.

Reference is made to U.S. Pat. No. 6,531,578 in which monoclonal antibodies are described that are specific for the recognition of iNOS in humans without cross-reacting with human eNOS or nNos. U.S. Pat. No. 6,531,578 is incorporated by reference in whole to the present application. An immunoassay using such monoclonal antibodies is capable of detecting iNOS in the blood which indicates the presence of sepsis within a very short period of time, a matter of minutes, when compared to the prior art tests which required several days to complete and obtain results. If sepsis is treated aggressively after recognition of its existence, persons afflicted with sepsis have a much better chance of recovering and surviving. Treatment of sepsis has been limited to known antibacterial, antifungal, and antiviral treatments. Such treatments have achieved limited success even with the rapid recognition of the presence of sepsis in a human. Although the relatively quick detection of sepsis, severe sepsis, or septic shock in humans permits the treatment of humans using conventional therapies, it is important to research and discover new and improved therapies for the treatment of these life threatening illnesses.

U.S. patent application Ser. Nos. 10/849,768 and 11/129,452 describe therapies of this type which were developed following the analysis of more than 1200 plasma samples obtained from ICU patients with sepsis or severe sepsis or at risk for developing sepsis as part of a clinical study for a potential new diagnostic for sepsis. The presence of iNOS in the plasma of patients with sepsis and those who would become septic within the next 48 hours was discovered. Also, particulate or vesicle associated iNOS was discovered in the plasma of septic patients. Neither soluble nor particulate iNOS was found in normal controls or in ICU patients who were not septic, such as trauma patients that often display similar physiological signs and symptoms as septic patients. It was reasoned that one or perhaps both forms of iNOS, i.e. soluble iNOS and/or particulate iNOS, might be partially responsible for the pathophysiology of sepsis

Based upon these observations, it was hypothesized (1) that if soluble and/or particulate iNOS contributed to the pathology of sepsis, then that form of iNOS would have to bind to cells and tissues through some receptor mediated process in order to exert its deleterious effects, and (2) that inhibition of the cellular binding, such as by a receptor antagonist, might be a beneficial therapy for the treatment of patients with or at risk for developing SIRS, sepsis, severe sepsis and septic shock.

An article entitled “Cloning and Characterization of Inducible Nitric Oxide Synthase from Mouse Macrophages”, Xie et al, Science, 256: 225-228 (1992), reported the cloning and isolation of iNOS. The iNOS enzyme was described as a soluble cytoplasmic protein.

Subsequently, articles entitled “Nitric Oxide: Novel Biology with Clinical Relevance”, Billiar, Ann Surg, 221#4: 339-349 (1995); “Nitric Oxide: Pathophysiological Mechanisms”, Gross et al, Annu Rev Physiol, 57: 737-769 (1995); “The Cell Wall Components Peptidoglycan and Lipoteichoic Acid from Staphylococcus Aureus Act in Synergy to Cause Shock and Multiple Organ Failure”, De Kimpe et al, Proc Natl Acad Sci USA, 92: 10359-10363 (1995); “Mechanism of Gram-Positive Shock: Identification of Peptidoglycan and Lipoteichoic Acid Moieties Essential in the Induction of Nitric Oxide Synthase, Shock, and Multiple Organ Failure”, Kengatharan et al, J Exp Med, 188#2: 305-315 (1998); and “Induction of Nitric Oxide Synthase in RAW 264.7 Macrophages by Lipoteichoic Acid from Staphylococcus aureus: Involvement of Protein Kinase C— and Nuclear Factor-KB-Dependent Mechanisms”, Kuo et al, J Biomed Sci, 10: 136-145 (2003), point to the fact that the lipopolysaccharide (LPS) cell-wall component of gram-negative bacteria, the lipoteichoic acid and peptidoglycan cell-wall components of gram-positive bacteria, fungi, and viruses can induce iNOS expression in vivo and in vitro in a wide variety of cell types.

Articles entitled “Mechanisms Of Suppression Of Macrophage Nitric Oxide Release By Transforming Growth Factor Beta”, Vodovotz et al, J Exp Med, 178#2: 605-613 (1993); “Vesicle Membrane Association Of Nitric Oxide Synthase In Primary Mouse Macrophages”, Vodovotz et al, J Immunol, 154#6: 2914-2925 (1995); and “Bladder Instillation And Intraperitoneal Injection Of Escherichia coli Lipopolysaccharide Up-Regulate Cytokines And iNOS In Rat Urinary Bladder”, Olsson et al, J Pharmacol Exp Ther, 284#3: 1203-1208 (1998), have shown that since the discovery of iNOS in mouse macrophages, its intracellular location is not exclusively in the cytosol. Vesicle-associated iNOS has been recognized and reported.

Articles entitled “Caveolin-1 Down-Regulates Inducible Nitric Oxide Synthase Via The Proteasome Pathway In Human Colon Carcinoma Cells”, Felley-Bosco E et al, Proc Natl Acad Sci USA, 97#26: 14334-14339 (2000); “Macrophage Nitric Oxide Synthase Associates With Cortical Actin But Is Not Recruited To Phagosomes”, Infect Immun, Webb J L et al, 69#10: 6391-6400 (2001); “Epithelial Inducible Nitric-Oxide Synthase Is An Apical EBP50-Binding Protein That Directs Vectorial Nitric Oxide Output”, Glynne P A et al, J Biol Chem, 277#36: 33132-33138 (2002); “Caveolin-1-Mediated Post-Transcriptional Regulation Of Inducible Nitric Oxide Synthase In Human Colon Carcinoma Cells”, Felley-Bosco E, Biol Res, 35#2: 169-176 (2002); “Heat Shock Protein 90 As An Endogenous Protein Enhancer Of Inducible Nitric-Oxide Synthase”, Yoshide M et al, J Biol Chem, 278#38: 36953-36958 (2003); “Protein Interactions With Nitric Oxide Synthase: Controlling The Right Time, The Right Place, And The Right Amount Of Nitric Oxide”, Kone B C et al, Am J Physiol Renal Physiol, 285#2: F178-F190 (2003); and “Protein-Protein Interactions Involving Inducible Nitric Oxide Synthase”, Zhang W et al, Acta Physiol Scand, 179#2: 137-142 (1997), have reported that when induced cells are lysed and fractionated by centrifugation, iNOS is found in the particulate fraction, and that iNOS has been found associated with a number of other proteins through protein-protein interaction. Such protein-protein interactions (other proteins associated with iNOS) include cortical actin, EBP 50 (ezrin-redixin-moesin-binding phosphoprotein 50), caviolin-1, Hsp90 (heat shock protein 90), kalirin, NAP110 (NOS-associated protein 1.10 kd), and Rac-GTPases. These protein-protein interactions have been found to localize iNOS to specific regions or structures within cells. Upon cell lysis and fractionation by centrifugation, either through vesicle association or by protein-protein interaction, a portion of the supposedly soluble iNOS protein has been shown to partition into the particulate fraction.

U.S. patent application Ser. No. 09/628,585, revealed the fact that iNOS found free in the liquid portion of the blood of a patient, i.e. plasma, indicates such patient has sepsis or will develop sepsis within the next 24 to 48 hours. Using a very sensitive chemiluminescent sandwich enzyme immunoassay (EIA), such plasma iNOS can be used as a very specific biochemical marker for the onset of sepsis. The heretofore referenced chemiluminescent sandwich (EIA) was based upon two of the anti-iNOS monoclonal antibodies (MAbs) of a panel of anti-iNOS antibodies which are disclosed in U.S. Pat. No. 6,531,578, mentioned heretofore.

U.S. patent application Ser. Nos. 10/849,768 and 11/129,452 propose therapeutic agents and treatments for iNOS in order to alleviate the sepsis, severe sepsis, or septic shock pathology in a mammalian subject, and U.S. patent application Ser. No. 11/208,143 describes a mammalian model of sepsis that can be used to evaluate potential therapeutic treatments of SIRS, sepsis, severe sepsis, or septic shock in a mammalian subject.

Although the detection of iNOS in the blood of patients may greatly aid in the treatment of those patients by conventional therapies and although other potential therapeutic treatments have been proposed and tested, the efficacy of such potential new therapeutics in treating humans has not yet been established.

An improved therapy for the treatment of sepsis, severe sepsis, and septic shock would be a notable advance in the medical field.

### BRIEF SUMMARY OF THE INVENTION

In accordance with the present invention a novel and useful therapeutic agent for the treatment of SIRS, sepsis, severe sepsis, or septic shock based upon inhibition of the binding to cellular receptors and inhibition of tissue uptake by receptor antagonists for particulate iNOS is herein provided.

Using a mammalian model of sepsis, severe sepsis, and septic shock, the ability to protect such mammalian subjects from lethal doses of particulate iNOS was explored. In one aspect of the present invention, the therapeutic agent of the present invention was administered to the mammalian subjects prior to the introduction of a particulate fraction of iNOS. In controlled experiments, it was determined that the therapeutic agent of the present invention blocked the binding of the lethal particulate fraction of iNOS to the cells presumptively through a cell receptor. It was also determined, that the supernatant fraction of iNOS served as the therapeutic agent in this regard. Further experimentation determined that fractionation by column chromatography of the supernatant fraction of iNOS produced (1) an unsedimented particulate iNOS fraction that augmented the lethal effects of the particulate iNOS which was used to challenge the mice subject to the mammalian model of sepsis, and (2) a soluble iNOS fraction that was protective against a lethal challenge of particulate iNOS by competing for binding to cellular receptors. In other words, purified soluble iNOS was found to protect the mammalian subjects from the lethal effects of particulate iNOS in vivo, through the action of the purified soluble iNOS functioning as a receptor antagonist.

Further tests employing radio-iodinated techniques confirmed that the protective effect of the soluble iNOS fractions was receptor mediated. Specifically, the initial increase in uptake of the iodinated tracers by the heart, spinal cord, and intestines of mammalian subjects was decreased (antagonized) by the administration of the soluble iNOS fractions. Consequently, it was shown that mammalian subjects were rescued from death from sepsis by use of the soluble fraction of iNOS as a cellular receptor antagonist to the lethal particulate fraction of iNOS. Such receptor antagonist may take the form of intact soluble iNOS, a fragment of soluble iNOS, or an analogue of soluble iNOS. In any case, the receptor antagonist of the present invention binds to the receptor and successfully competes with the lethal particulate iNOS for binding to the receptor.

It may be apparent that a novel and useful therapeutic agent and method for the treatment of illnesses in mammalian subjects where iNOS is present has been described.

It is therefore an object of the present invention to provide a therapeutic agent and method for the treatment of mammalian illnesses in which iNOS is present by the use of a cellular receptor antagonist.

Another object of the present invention is to provide a therapeutic agent and method for the treatment for an illness in a mammalian subject in which iNOS is present employing a cellular receptor antagonist in the form of the soluble fraction of iNOS.

It is another object of the present invention to provide a cellular receptor antagonist to the lethal effects of the particulate fraction of iNOS in the form of a soluble fraction of iNOS which is most effective in its purified form.

A further object of the present invention is to provide a treatment for sepsis related diseases in mammalian subjects using a receptor antagonist which exhibits activity in at least the heart, spinal cord, and intestines of the mammalian subject.

Yet another objective of the present invention is to provide a receptor antagonist which successfully competes with particulate iNOS in the form of intact soluble iNOS, a fragment of soluble iNOS, or an analogue of soluble iNOS to treat systemic inflammatory response syndrome (SIRS), sepsis, severe sepsis and/or septic shock in a mammalian subject.

The invention possesses other objects and advantages especially as concerns particular characteristics and features thereof which will become apparent as the specification continues.

### BRIEF DESCRIPTION OF THE SEVERAL VIEWS OF THE DRAWING

FIG. 1 is a photograph showing a field of peripheral blood mononuclear cells (PBMCS) from a septic patient, in which iNOS-containing vesicles (presumably apoptotic bodies) are separate from the cells, at 200×.

FIG. 2 is an image of a western immunoblot showing the supernatant (soluble) fraction and particulate fraction of iNOS where, lanes 1 and 7 are molecular weight standards, lane 2 is the induced supernatant (soluble) fraction at 5μl, lane 3 is the induced soluble fraction at 2.5μl, lane 4 is the induced particulate fraction at 5 μl, lane 5 is the induced particulate fraction at 2.5μl, and lane 6 is an iNOS standard.

FIG. 3 is a chart illustrating the 48 hour survival of mice primed with LPS and four hours later administered the chemical entities described in Example I.

FIG. 4 is a chart illustrating the seven day survival of mice primed with LPS and four hours later administered the chemical entities described in Example II.

FIG. 5 is a chart illustrating the seven day survival of mice primed with LPS and four hours later administered certain chemical entities described in Example III.

FIG. 6 is an image of a Western blot showing molecular weight standards (lane 1); intact iNOS contained in the high speed supernatant (lanes 2 and 3); intact iNOS and two fragments of iNOS contained in the low speed particulate fraction (lanes 4 and 5); and no iNOS contained in the high speed particulate fraction (lanes 6 and 7) as described in Example IV.

FIG. 7 is a chart summarizing the results of Example V demonstrating the lethality of particulate iNOS with or without LPS priming of the challenged mice and the ability of anti-iNOS monoclonal antibody 2A1-F8 to rescue mice that would otherwise die of sepsis.

FIG. 8 is an image of a Western blot of fractions 9-18 (lanes 1-10, respectively) collected from a Sephadex G-200 column chromatography of the supernatant fraction of iNOS obtained from induced and lysed DLD-1-5B2 cells as described in Example IX, where fractions #11 and 12 (lanes 3 & 4) comprise Peak #1, eluted at the void volume of the column, and contained residual, unsedimented particulate iNOS and fragments of iNOS present in the supernatant fraction of iNOS used as starting material; fractions #17 and 18 (lanes 9 & 10) comprise Peak #2, eluted approximately 25 ml later than peak #1 from the G-200 column, and contained partially purified intact soluble iNOS and fragments of iNOS that were unseparated from the intact iNOS by this chromatography procedure.

FIG. 9 is a chart illustrating the seven (7) day survival of LPS-primed mice challenged with the chemical entities as described in Example IX.

FIG. 10A is a bar chart that displays the results obtained for heart in Example X.

FIG. 10B is a bar chart that displays the results obtained for the spinal cord in Example X.

FIG. 10C is a bar chart that displays the results obtained for the intestines in Example X.

FIG. 10D is a bar chart that displays the results obtained for the liver in Example X.

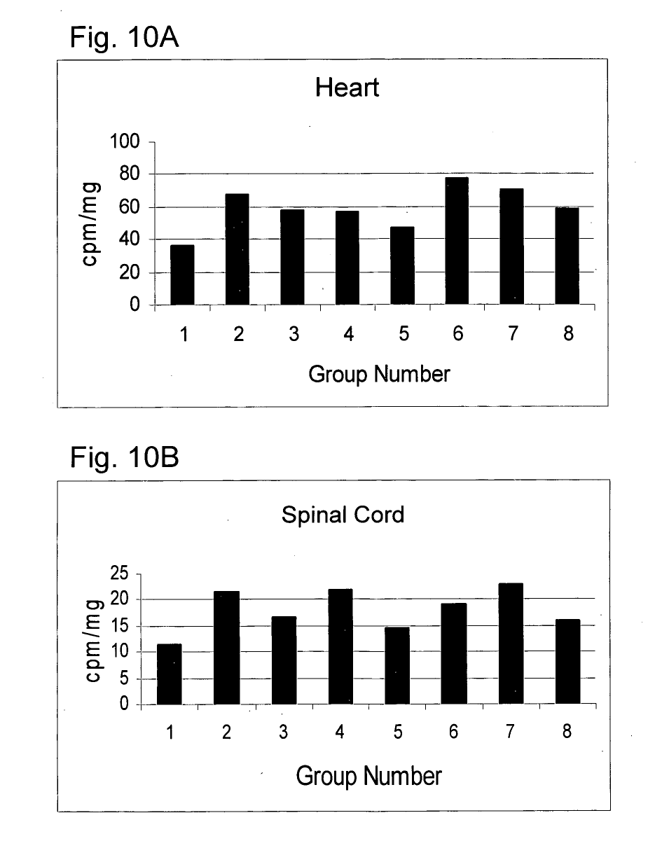

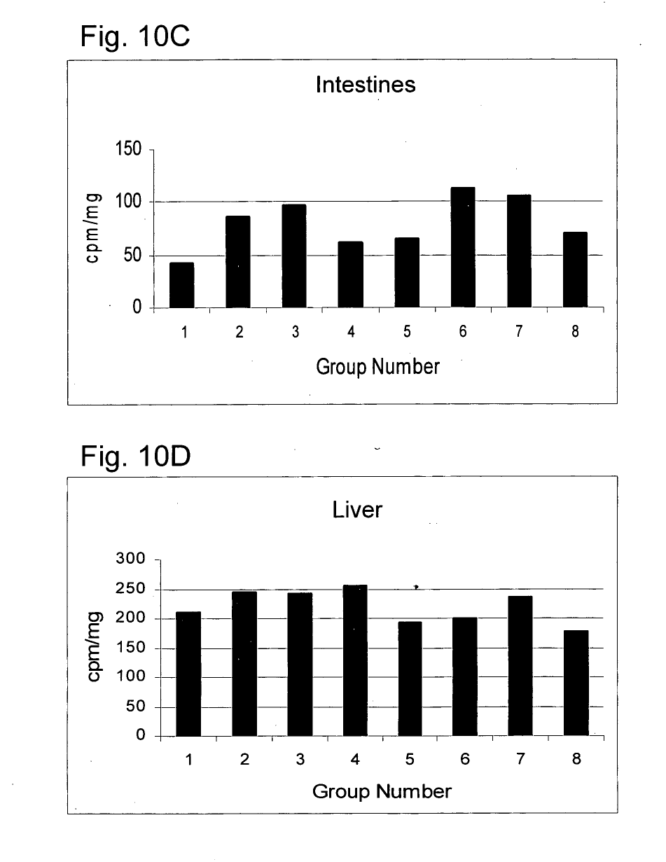

The groups in FIGS. 10A, 10B, 10C and 10D: 1 & 5 received saline at T=0 hr+saline at T=3.5 hr treatment, groups 2 & 6 received LPS at T=0 hr+saline at T=3.5 hr treatment, groups 3 & 7 received LPS at T=0 hr+unlabeled Fr. #12 at T=3.5 hr treatment, and groups 4 & 8 received LPS at T=0 hr+unlabeled Fr. #17 at T=3.5 hr treatment. With respect to each FIG. 10A-10D, groups 1-4 were administered 4×10⁶ cpm of ¹²⁵I—Fr12 and groups 5-8 were administered 4×10⁶ cpm of ¹²⁵I—Fr17 four hours (T=4 hr) after the start of the experiment. All animals were euthanized at T=5 hr, organs dissected, weighed and counted for their content of ¹²⁵I.

For a better understanding of the invention reference is made to the following detailed description of the preferred embodiments thereof which should be taken in conjunction with the prior described drawings.

### DETAILED DESCRIPTION OF THE PREFERRED EMBODIMENTS OF THE INVENTION

Various aspects of the present invention will evolve from the following detailed description of the preferred embodiments thereof and examples which should be taken together with the hereinbefore described drawings.

In clinical trials, more than 340 human subjects were enrolled, and over 1,200 blood samples were collected and analyzed to determine if the chemilumenescent EIA for iNOS described in U.S. Pat. No. 6,531,578 and U.S. patent application Ser. No. 09/628,585 prognosticated the onset of sepsis and monitored the course of the pathology. It was found that free iNOS (soluble iNOS) was present in the blood samples of ICU patients with sepsis or who would develop sepsis over the next 24-48 hours, but not in the blood of normal control or non-septic ICU patients. Also, particulate iNOS, in the form of membrane associated iNOS, vesicle-associated iNOS, or iNOS in association with another protein (by protein-protein interaction), was present in blood samples from septic patients, but not in blood samples from normal controls or non-septic patients. The vesicles that contained the particulate iNOS were not attached to any of the cells.

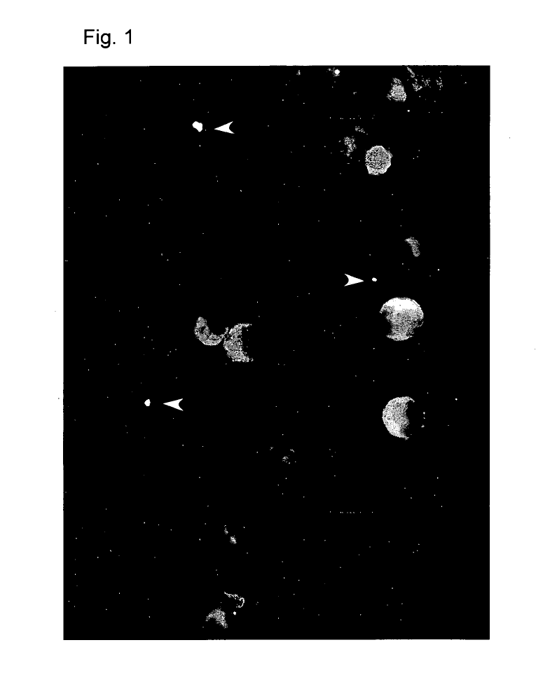

FIG. 1 shows the presence of human iNOS in peripheral blood mononuclear cells (PBMCs) and in vesicles/globules that are not cell associated. In FIG. 1 small extra cellular vesicles, presumably apoptotic bodies, appear as white dots and are separate from the cells (arrows). The immunostaining for iNOS with the anti-iNOS monoclonal antibody 2A1-F8 appears granular in the PBMCs. The anti-iNOS monoclonal antibody 2A1-F8 is disclosed in U.S. Pat. No. 6,531,578. FIG. 1 provides evidence for the existence of apoptotic bodies in vivo in humans. Particulate or vesicle-associated iNOS was only found in samples from patients afflicted with sepsis, severe sepsis, or septic shock. The presence of apoptotic bodies as revealed in FIG. 1 in the blood stream of a human may be an indication of the presence of sepsis or an indication of the severity of the pathology.

Since soluble iNOS and the particulate or vesicle-associated iNOS are only found in the blood of critically ill patients, the contribution to the pathology of SIRS (pre-sepsis), sepsis, severe sepsis, or septic shock by these forms of iNOS was investigated. In other words, the presence of soluble or particulate iNOS in the circulation was theorized to be deleterious to patients with sepsis or pre-sepsis. Consequently, it was reasoned that blocking cellular binding and tissue uptake of the deleterious form(s) of iNOS might be a possible therapeutic treatment for such illnesses.

In order to gather data that might confirm such a hypothesis, a mouse model of sepsis was used in a series of experiments. Tests were employed to determine (1) whether or not the supernatant fraction and/or particulate fraction of iNOS obtained from induced and lysed DLD-1-5B2 cells contributed to the pathology of sepsis, severe sepsis, or septic shock and (2) whether or not inhibition of cellular binding of the deleterious form(s) of iNOS could be blocked by receptor antagonists as possible therapeutic treatments for these life threatening conditions.

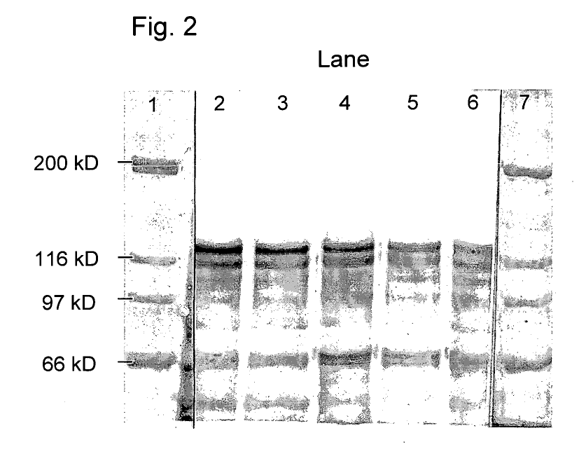

As previously stated, DLD-1-5B2 cells can be induced to produce human iNOS by the addition of a mixture of cytokines. In an article entitled “Transcriptional Regulation of Human Inducible Nitric Oxide Synthase Gene In An Intestinal Epithelial Cell Line”, Linn et al, Am J Physiol, 272: G1499-G1508 (1997), it was shown that DLD-1 cells can be induced to produce human iNOS. Also, when the induced cells are lysed, two types of iNOS can be isolated by centrifugation, a supernatant (soluble) iNOS fraction and a particulate iNOS fraction. FIG. 2 shows a Western immunoblot which indicates that the pooled supernatant (soluble) fraction of induced DLD-1-5B2 (a clone of DLD-1) cells contained iNOS (lanes 2 and 3) at the predicted molecular weight of 131 kD. Also, the particulate fraction of induced DLD-1-5B2 cells likewise contained iNOS (lanes 4 and 5) as shown by the band at 131 kD. Lane 6 contained an iNOS standard and lanes 1 and 7 contained standard proteins used as molecular weight markers at the indicated weights. In addition to intact iNOS, both the supernatant (soluble) fraction of iNOS (lanes 2 and 3) and the particulate fraction of iNOS (lanes 4 and 5) were found to contain fragments of iNOS to which anti-iNOS specific monoclonal antibody 2D2-B2 bound, FIG. 2. In order to produce and isolate the supernatant and particulate fractions of iNOS from induced DLD-1-5B2 cell cultures, the following steps were followed:

1. DLD-1-5B2 cells were grown in culture starting from frozen cryopreserved cells;

2. The expression of iNOS was induced in the cells;

3. The induced cells were harvested; and

4. The iNOS in the induced cells was isolated and fractionated into soluble and particulate fractions.

Briefly, cryopreserved cells were transferred into a T-75 flask containing DLD-1-5B2 medium (90 percent DMEM and 10 percent FBS supplemented with Pen/Strep), and cultured in a humidified atmosphere of 5 percent CO₂ in air at 37° C. The culture medium was changed every other day until the cells were almost confluent. Subsequently, the medium was changed daily until the cell cultures were either split or induced. When the DLD-1-5B2 cells were near confluence and in log-phase growth, the cells were split 1:6 to 1:10 into additional T-75 flasks.

DLD-1-5B2 cells two days past confluence were induced to express human iNOS using a mixture of recombinant human gamma interferon (rhIFNγ) at 8.33 ng/ml, recombinant human tumor necrosis factor-alpha (rhTNFα) at 3.3 ng/ml, and recombinant human interleukin-1β (rhIL-1β) at 3.3 ng/ml for 12-18 hours before being washed and harvested with trypsin/EDTA. The induced cells were washed three times with sterile PBS, were transferred into sterile tubes in a small volume of sterile PBS, and stored frozen at −20° C. until processed for the isolation and fractionation of iNOS.

The frozen, induced DLD-1-5B2 cells were thawed in an ice water bath and lysed by two rapid freeze/thaw cycles using dry ice. The iNOS produced by the induced DLD-1-5B2 cells was then fractionated into a supernatant and a particulate fraction by centrifugation at 16,000×g at 4° C. for 30 minutes to sediment the particulate fraction. The supernatant fraction that contained the soluble iNOS and some unsedimented particulate iNOS was transferred and stored at −20° C. or used fresh. The pellets were washed once, resuspended in a small volume of ice cold sterile PBS, and the resulting particulate fraction of iNOS was stored at −20° C. or used fresh.

In general, once the cryopreserved cells were thawed and placed in culture medium, they reach log phase growth after a few days at 37° C. in a humidified 5 percent CO₂/95 percent air atmosphere. DLD-1 cells are available from ATCC (CAT.#CCL221). The DLD-1-5B2 cell line was derived by sub-cloning the DLD-1 cells using standard cloning techniques.

A mammalian model of sepsis, severe sepsis, and septic shock has been developed and tested. In particular, tests were employed to determine whether or not the supernatant and/or the particulate iNOS fractions contributed to the pathology of sepsis, severe sepsis, or septic shock. In order to gather data that might confirm such hypotheses, a mammalian model of sepsis, utilizing mice, was used in a series of experiments. The effects of the supernatant iNOS fraction and of the particulate iNOS fraction were tested on mice primed (1) with a sub-lethal dose of the innate immune system activator LPS or (2) without priming (a saline injection) as a model of sepsis. This was done to determine the effects that the two different fractions of iNOS protein had on the mice. The results of these experiments led to the discovery that the iNOS protein plays a role in the pathophysiology of sepsis in the mice. Several experiments were conducted, and it was discovered that iNOS in the particulate fraction, rather than the supernatant fraction of iNOS, plays a role in causing death in this mouse model of sepsis. It was also found that the particulate or vesicle associated iNOS alone was lethal and did not require a priming dose of an innate immune system activator for it to be lethal. However, priming with an innate immune system activator augmented the effect, and thereby, required a lower dose of the particulate iNOS for lethality. Administration of the particulate iNOS fraction to non-primed mice could result in almost immediate death depending upon the dose administered. Particulate iNOS is believed to be responsible for the lethal effect observed both in LPS primed mice and non-primed mice.

In summary, the effects of the supernatant iNOS fraction and of the particulate iNOS fraction were tested on non-primed mice and on mice primed with a sub-lethal dose of LPS as a model of sepsis. This was done to determine the effects the two different fractions of iNOS protein had on the viability of the mice. The results of these experiments led to the discovery that the iNOS protein plays a role in the pathology of sepsis. Several experiments were conducted, and it was discovered that iNOS in the particulate fraction, rather than the supernatant fraction of iNOS, plays a role in causing death in this mouse model of sepsis. It is also found from these experiments that LPS priming of mice was not necessary for the effect of the particulate iNOS to be exerted. The administration of the particulate iNOS fraction to non-primed mice caused almost immediate death. Consequently, particulate iNOS is believed to be responsible for the lethal effect observed in LPS primed mice.

It was reasoned that the lethal effect(s) exerted by the particulate iNOS must have resulted from cellular binding in various organs. We hypothesized (1) that cellular binding of the deleterious form(s) of iNOS was receptor mediated; (2) that cellular binding could be quantitated by measuring organ uptake of ¹²⁵I-labeled iNOS; and (3) that cellular binding could be competitively inhibited by a receptor antagonist which would decrease the amount of uptake of ¹²⁵I-labeled iNOS by an organ since the receptor antagonist would bind to and occupy the receptor's binding site, and thereby, decrease the quantity of ¹²⁵I-labeled iNOS that could be bound. These hypotheses were tested in a series of experiments.

The following examples are illustrative of the present invention and are not deemed to limit the scope of the present invention.

### EXAMPLE I

The two fractions of human iNOS, illustrated in FIG. 2, were investigated as to their effect on LPS primed mice as an animal model of sepsis. Prior to starting the experiment, intact soluble iNOS and fragments of iNOS were removed from the supernatant fraction by selective absorption onto MAG-BEADS coated with one or more of the anti-iNOS MAbs found in the U.S. Pat. No. 6,531,578. Briefly, MAG-BEADS covalently bonded to goat anti-mouse IgG IgG were purchased from the Pierce Chemical Co. in Rockford, Ill. Culture supernatant containing secreted anti-iNOS MAbs from clones 21C10-1D10, 2A1-F8, 1E8-B8 and 2D2-B2 were applied individually to aliquots of the suspended MAG-BEADS in order to load the MAG-BEADS with monoclonal antibodies specific for iNOS. The supernatant fraction containing intact soluble iNOS and fragments of iNOS were diluted 1:2 and applied to pooled, washed, and resuspended anti-iNOS coated MAG-BEADS. The suspension was incubated overnight with gentle mixing to allow the soluble iNOS and fragments of iNOS to bind to the anti-iNOS MAbs coated onto the MAG-BEADS before the tube containing the suspension was placed onto a magnetic rack. All the beads congregated on the sides of the tube next to the magnets. The resulting iNOS-depleted supernatant fraction was transferred and diluted to a final volume to yield a 1:5 dilution as compared to the stock supernatant fraction. A 1:5 dilution of the stock iNOS supernatant fraction was also prepared in sterile saline. Samples of the 1:5 iNOS-depleted supernatant fraction, of the 1:5 diluted stock supernatant fraction, and of the iNOS coated MAG-BEADS used to create the iNOS-depleted supernatant fraction were all analyzed to determine if the intact soluble iNOS and fragments of iNOS had been removed and to demonstrate that the intact soluble iNOS and fragments of iNOS were bound to the anti-iNOS MAbs attached to the MAG-BEADS. These analyses showed that more than 90 percent of the soluble iNOS and of the fragments of iNOS had been removed from the supernatant (soluble) fraction (iNOS-depleted soluble fraction), and that the iNOS and iNOS fragments were bound to the MAG-BEADS which had been loaded with the anti-iNOS MAbs.

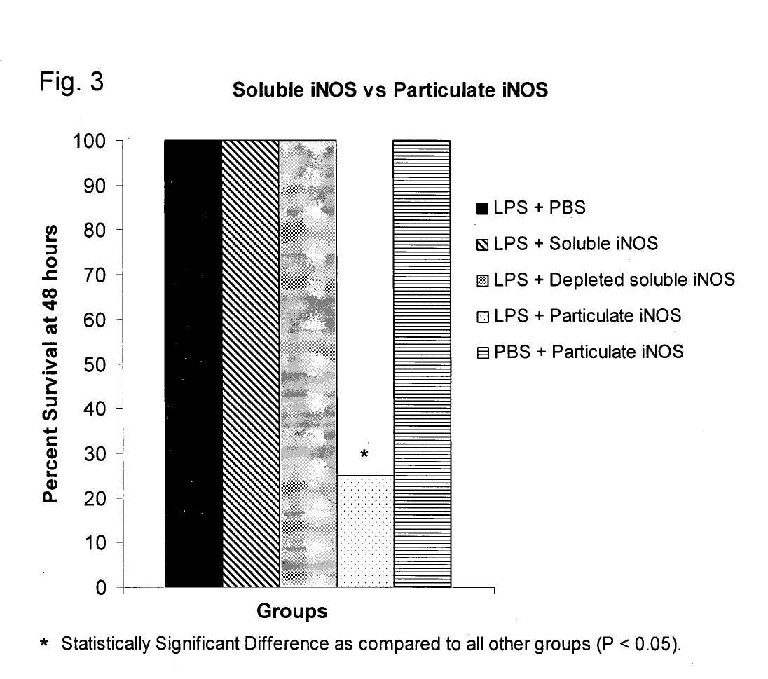

Groups of mice containing both genders were injected IP with sterile saline only, or with a sub-lethal dose of LPS (2 mg/kg body weight of LPS Serotype 0111:B4 from E. coli, obtained from Sigma Chemical Co., Saint Louis, Mo.) in sterile saline. After four hours, only the mice injected with LPS became lethargic and developed diarrhea. The saline and LPS-primed mice were then given a tail vein injection of either saline or one of the following: the supernatant fraction containing iNOS (soluble iNOS), the supernatant fraction depleted of iNOS (iNOS-depleted soluble fraction), or a suspension of particulate iNOS produced by and isolated from induced DLD-1-5B2 cells. FIG. 3 shows the results of this experiment. None of the saline-primed mice showed any effect with any of the test samples. No effect was seen with the LPS-primed mice upon administration by tail vein injection of either a dose of saline, a dose of the supernatant fraction of iNOS, or a dose of iNOS-depleted supernatant fraction. However, when the particulate fraction of iNOS was administered to the LPS prime mice, all the mice died almost immediately. In this series of experiments, no effect was seen on the saline-primed mice given the same dose of particulate iNOS. It was concluded that the particulate fraction of iNOS, but not the supernatant fraction of iNOS, when administered to LPS-primed mice caused an almost immediate death.

### EXAMPLE II

The anti-human iNOS MAbs found in U.S. Pat. No. 6,531,578 were employed in order to investigate the inhibition of the killing effect seen with the particulate fraction of human iNOS in LPS-primed mice. Particulate iNOS and fragments of iNOS contained in the particulate fraction were removed from the particulate fraction by selective absorption onto MAG-BEADS coated with the anti-iNOS MAbs as described in Example I. A similar procedure to the one described with respect to depletion of the supernatant fraction in Example I was employed. Samples of the iNOS-depleted particulate fraction, of the stock particulate fraction, and of the iNOS loaded MAG-BEADS used to create the iNOS-depleted particulate fraction, were analyzed to show that the particulate iNOS and iNOS fragments had been removed. Our analyses showed that more than 90 percent of the particulate iNOS and iNOS fragments had been removed from the particulate fraction (iNOS-depleted particulate fraction) and that the iNOS was bound to the MAG-BEADS which had been loaded with the anti-iNOS MAbs.

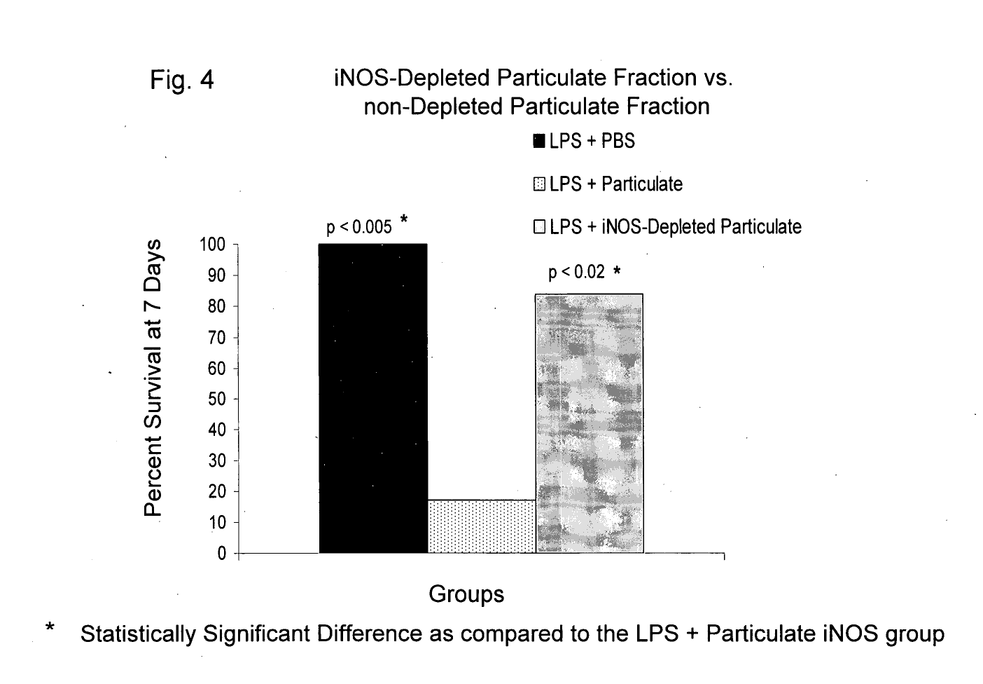

The effect that the iNOS-depleted particulate fraction had on the LPS-primed mice was compared to that seen with the stock (non-depleted) particulate fraction containing particulate iNOS. Groups of mice containing both genders were injected IP with a sub-lethal dose of LPS (2 mg/kg body weight of LPS serotype 0111:B4 from E. coli obtained from the Sigma Chemical Company) in sterile saline. After four hours, all the mice primed with LPS became lethargic and developed diarrhea. The various groups of mice were then given a tail vein injection of either saline, stock particulate iNOS at a 1:10 dilution, or iNOS-depleted particulate fraction at a 1:10 dilution as compared to the starting stock suspension. FIG. 4 represents these definitive results. None of the mice that received a priming IP injection of LPS followed four hours later by a tail injection of saline showed any effect since they all survived seven days until the end of the experiment of this Example. However, only 17 percent (one out of six) of the mice that received a priming IP injection of LPS followed four hours later by a tail injection of particulate iNOS at a 1:10 dilution, survived for seven days. Significantly, 84 percent (five of six) of the LPS-primed mice that received a tail vein injection of the iNOS-deleted particulate fraction survived for seven days. When these data were compared, a high degree of statistically significant difference was found between the survival of the mice administered the particulate iNOS fraction and those administered saline (P<0.005 by Student's T-test) or the iNOS-depleted particulate fraction (P<0.02). There was no statistically significant difference between the LPS-primed mice that received a saline IV injection and those that received the iNOS-depleted particulate fraction. Thus, the specific removal of the particulate iNOS and the fragments of iNOS from the particulate fraction abolished the lethal effect seen in the LPS-primed mice that received the particulate iNOS fraction. It was concluded that (1) particulate iNOS, fragments of iNOS in the particulate fraction, or particulate iNOS in association with one or more proteins was responsible for the lethal effect observed in LPS-primed mice and (2) removal of the particulate iNOS, iNOS fragments, or particulate iNOS in association with one or more proteins, by absorption from solution using immobilized anti-iNOS MAbs, stopped the lethal effects asserted by the administration of the particulate iNOS.

### EXAMPLE III

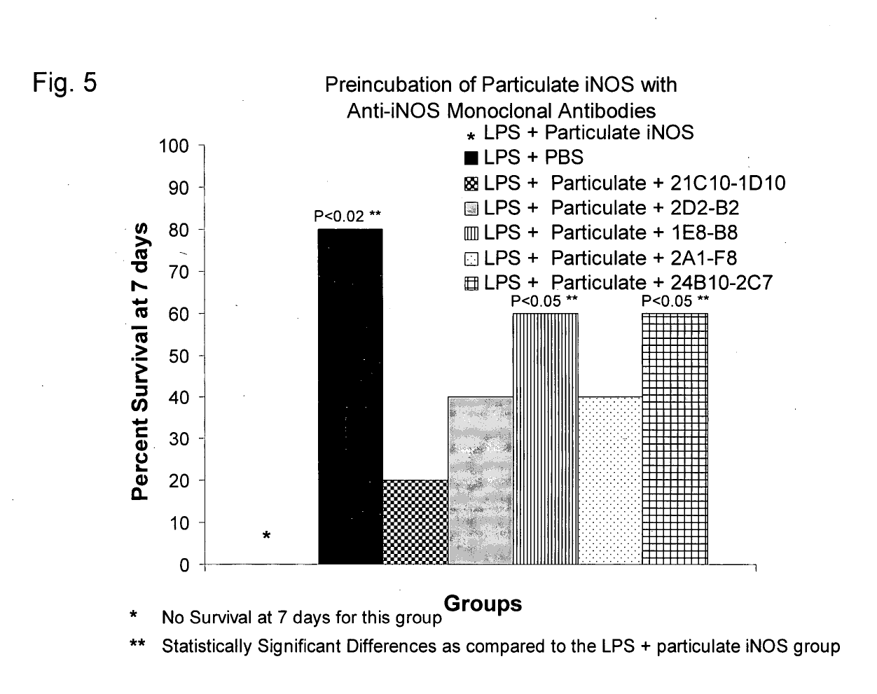

A second method was employed to study the ability of the anti-human iNOS MAbs of U.S. Pat. No. 6,531,578 to inhibit the killing effect seen with the particulate fraction of human iNOS in LPS-primed mice as a model for sepsis. Instead of physically removing the particulate iNOS and iNOS fragments from the particulate fraction as was performed in Example II, individual anti-iNOS MAbs contained in ascites fluid were added directly to aliquots of the particulate fraction and allowed to bind prior to use. The proteins contained in the particulate iNOS fraction were allowed to bind to the anti-iNOS MAbs for 45 minutes before the material was injected IV into mice. Five different anti-iNOS MAbs were tested for their individual ability to inhibit (neutralize) the killing effect of the particulate human iNOS. Groups of mice were primed with a sub-lethal dose of LPS (2 mg/kg body weight of LPS Serotype 0111:B4 from E. coli obtained from the Sigma Chemical Company) in sterile saline. After four hours all the LPS-primed mice became lethargic and developed diarrhea. The various groups of mice were given a tail vein injection of either saline, stock particulate iNOS at a 1:10 dilution, or stock particulate iNOS at a 1:10 dilution that had been preincubated for 45 minutes with one of five different anti-iNOS MAbs. Each of the five different anti-iNOS MAbs was used at a 1:50 dilution of the ascites fluid. The results varied and are shown in FIG. 5. All the LPS-primed mice that received a tail vein injection of the stock particulate iNOS diluted 1:10 in sterile saline died within the first 24 hours of the seven day experiment. In contrast, four out of five (80 percent) of the LPS-primed mice administered a saline tail vein injection survived seven days (P<0.02). The ability of the anti-iNOS MAbs to neutralize the lethal effect of the intact particulate iNOS and iNOS fragments varied depending on the MAb being tested. Of the five different anti-iNOS MAbs tested, anti-iNOS MAb 1E8-B8 and 24B10-2C10 were the best at neutralizing the lethal effects of the particulate iNOS on LPS-primed mice. In both cases, three out of five mice survived seven days (P<0.05). Two other anti-iNOS MAbs (2D2-B2 and 2A1-F8) were also somewhat effective in stopping the mice from dying, i.e. two out of five mice in each of these groups survived seven days. One anti-iNOS MAb (21C10-1D10) was much less effective since only one out of five mice survived seven days. It was concluded (1) that it is not necessary to remove physically the particulate iNOS or iNOS fragments from the solution in order to neutralize the lethality; (2) that anti-iNOS MAbs can neutralize the lethal effects of the particulate fraction of iNOS on LPS-primed mice by binding to the intact iNOS, to the iNOS fragments, or to the protein-protein complex containing particulate iNOS; and (3) that different anti-iNOS MAbs vary in their individual ability to neutralize the lethality of particulate iNOS either as particulate iNOS itself, or as a fragment of iNOS, or in association with one or more proteins.

### EXAMPLE IV

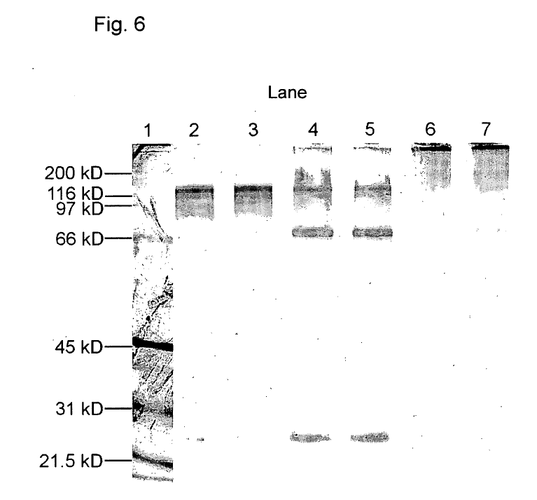

Experiments were performed to determine if the immunoreactive iNOS contained in the particulate fraction is located in the cellular membrane, or in vesicles, or in other structures, such as membrane fragments. Cytokine induced DLD-1-5B2 cells were lysed by hypotonic shock instead of by 2 freeze/thaw cycles in order to rupture the cellular membrane and to release their cellular components and membrane structures, including blebbing vesicles, into the solution and, thereby, avoid denaturing the proteins, as can occur with multiple freeze/thaw cycles. The solution containing the lysed cells was subjected first to low speed centrifugation (at 300×g) to obtain a low speed particulate pellet. The low speed supernatant was then subjected to higher speed centrifugation (at 16,000×g) to produce both a high speed particulate pellet and a high speed supernatant. When these three fractions were tested for their killing activity in LPS-primed mice, the lethal activity was only found in the low speed particulate fraction. Neither the high speed pellet nor the high speed supernatant was lethal when injected intravenously into LPS-primed mice. When the low speed particulate fraction was examined microscopically, two types of structures were observed. One was the cellular membrane of “ghost” cells, i.e. ruptured cell remnants, and the other was small vesicles which many times appeared linked together like beads on a string. When this preparation was stained by immunofluorescence using the anti-iNOS MAbs of U.S. Pat. No. 6,531,578 to immunolocalize the iNOS, intense fluorescent staining was observed exclusively in the small vesicles. No IFA staining of iNOS was observed in the “ghost” cells or in any other structure. The size of these vesicles (apoptoic bodies) and their intense IFA staining with the noted anti-iNOS MAbs is very similar, if not identical, to that observed in the blood of human septic patients (FIG. 1). When these preparations were also analyzed by Western blot after SDS-PAGE separation of the proteins (FIG. 6), the high speed supernatant was found to contain intact iNOS. The low speed particulate fraction contained intact iNOS, but it also contained two iNOS fragments that bound the anti-iNOS MAb 2D2-B2 used in these experiments (FIG. 6). It was repeatedly found that the high speed supernatant does not kill the LPS-primed mice in the animal model of sepsis of Examples I-III while the particulate fraction of iNOS whether produced by freeze/thaw cycles or by hypotonic shock is lethal to the LPS primed mice.

### EXAMPLE V

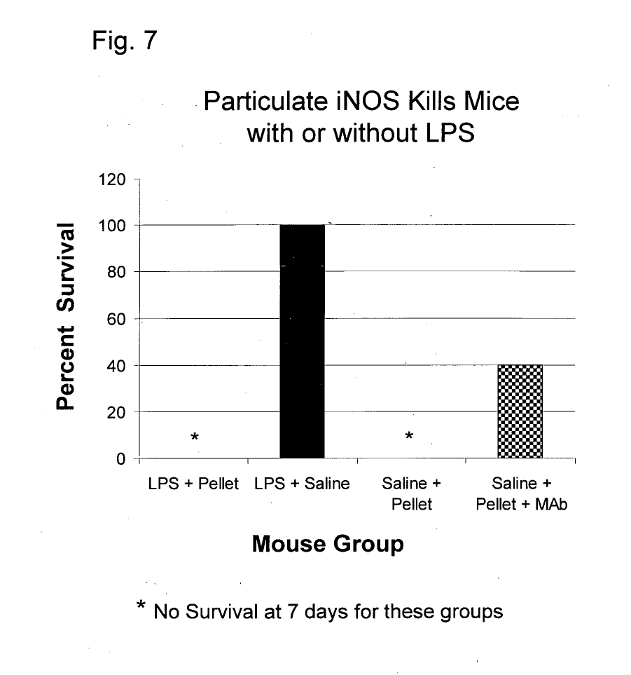

In a series of experiments the particulate fraction of iNOS, with or without LPS priming, was determined to kill mice, and treatment with anti-iNOS MAb was deemed capable of rescuing some mice that would otherwise have died. In these experiments mice were primed with LPS or saline for four hours before being challenged with either saline, the particulate fraction of iNOS isolated from induced DLD-1-5B2 cells, or the particulate fraction of iNOS that had been preincubated for 30 minutes with anti-iNOS MAb 2A12-A4. The particulate fraction of iNOS killed all five mice in both the LPS primed group and the group that had only been primed with saline. When the mice were challenged with saline instead of the particulate fraction of iNOS, after being primed with LPS, all five mice in the group lived. These data showed (1) that the dose of LPS used to prime the mice was not lethal and (2) that challenging the mice with the particulate fraction of iNOS with or without LPS priming resulted in death by sepsis. If the particulate fraction of iNOS was pretreated with anti-iNOS MAb, 40 percent of the mice (two of five) were rescued from death by sepsis. This showed that the anti-iNOS MAb 2A12-A4 is a neutralizing antibody and might be useful for therapeutic treatment of sepsis. The effectiveness of other potential treatments for sepsis can be evaluated using this animal model. FIG. 7 depicts the result of this example, and particulate iNOS is labeled “pellet” in FIG. 7.

### EXAMPLE VI

The effectiveness of the particulate fraction of iNOS to kill mice after priming with either LPS or saline was investigated in a number of experiments using different doses of the particulate fraction of iNOS. Mice were primed with either LPS or sterile saline four hours prior to the challenge with the particulate fraction of iNOS. Cumulative data indicated that of the 35 total mice in both groups, 24 died in the LPS primed group i.e. 68.6 percent died and 31.4 percent survived, and that 20 died in the saline primed group, i.e. 57.1 percent died and 42.9 percent survived. Statistical analysis of these data show that no significant difference exists between these two groups which means that the particulate fraction of iNOS is just as lethal by itself as it is after a priming dose of the innate immune system activator, lipopolysaccharide (LPS).

### EXAMPLE VII

In two series of experiments, (1) humanized MAb 1E8-B8 immobilized on Mag-beads, (2) peptide G-11 to which MAb 1E8-B8 binds, and (3) the particulate fraction of iNOS obtained from cytokine induced DLD-1-5B2 cells that were lysed either by 2 freeze/thaw cycles or by hypotonic shock were used to recover the hiNOS and associated materials including vesicles bound to the anti-hiNOS loaded MAG-BEADS. After treating the two particulate fractions with humanized MAb 1E8-B8 immobilized on MAG-BEADS, the MAG-BEADS were washed to remove unbound material. The material bound to the anti-hiNOS loaded MAG-BEADS was competed-off the humanized anti-hiNOS MAb by incubating the loaded beads with a high concentration (100 μg) of peptide G-11. The material competed-off the humanized anti-hiNOS 1E8-B8 MAb bound to the MAG-BEADS was centrifuged, washed and used to challenge mice primed with a sub-lethal dose of LPS in our mouse model of sepsis. Both samples were found to possess the lethal activity initially discovered in the particulate fraction of iNOS obtained from cytokine induced and lysed DLD-1-5B2 cells. Namely, with the recovered sample from the particulate fraction of iNOS prepared by 2 freeze/thaw cycles, 2 out of 2 mice died, and with the recovered sample from the particulate fraction of iNOS prepared by hypotonic shock, 1 out of 2 mice died. When the proteins contained in the material competed-off the anti-hiNOS loaded MAG-BEADS were analyzed by Western blots using anti-hiNOS MAb clone 2D2-B2 that binds to hiNOS[781-798], three main bands were found at apparent molecular weights of 131 kD, 70 kD, and 27 kD for the material recovered from the particulate fraction of iNOS prepared by 2 freeze/thaw cycles. A single band at 70 kD was found for the sample recovered from the low speed particulate fraction of iNOS prepared by hypotonic shock.

### EXAMPLE VIII

In a series of experiments the ability of the supernatant fraction of iNOS obtained from induced and lysed DLD-1-5B2 cells to protect LPS-primed mice from a challenged with a lethal dose of the particulate fraction of iNOS was explored. In this series of experiments mice were primed with a sub-lethal dose of LPS as previously described, and were then challenged four hours later with one of three different treatments: (group 1, N=5) the particulate fraction of iNOS from cytokine induced and lysed DLD-1-5B2 cells at a dose that kills all the mice (LD₁₀₀); (group 2, N=5) the particulate fraction of iNOS at an equivalent dose to group #1 plus a four times higher dose of the supernatant fraction of iNOS from cytokine induced and lysed DLD-1-5B2 cells, simultaneously administered; and (group 3, N=5) a four times higher dose of the supernatant fraction of iNOS from cytokine induced and lysed DLD-1-5B2 cells administered 30 minutes prior to the administration of the particulate fraction of iNOS. In these experiments, all the animals in groups 1 and 2 died. However, all the animals in group 3 survived (P<0.001 by Student's T-test). Thus, the prior administration of the supernatant fraction of iNOS was protective against a lethal challenge of the particulate fraction of iNOS. When administered prior to the particulate fraction, the non-lethal supernatant fraction blocked the lethal cellular events that resulted from the administration of the particulate fraction. It was concluded that the supernatant fraction of iNOS blocked the binding of the lethal material to cells presumably through a cellular receptor, and thereby protected the animals from a lethal dose of the particulate fraction of iNOS.

### EXAMPLE IX

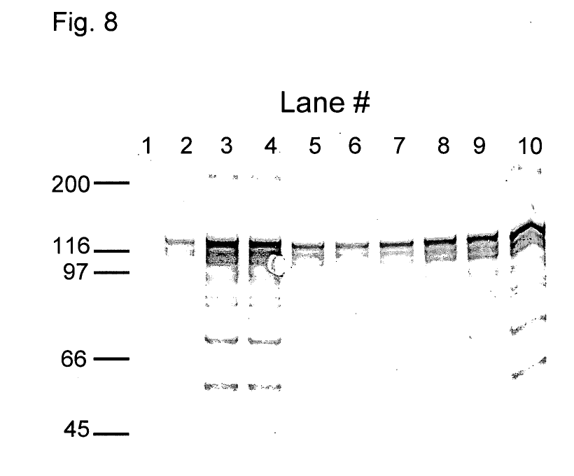

The supernatant fraction of iNOS obtained from induced and lysed DLD-1-5B2 cells was fractionated by column chromatography on Sephadex G-200 size exclusion gel. Two peaks of iNOS immunoreactive material eluded off the column, FIG. 8: Peak #1 at the void volume of the column (fractions #11 and 12) and Peak #2 approximately 25 ml later (fractions #17 and 18). The fractions that comprised Peak #1, fractions #11 and 12, were cloudy and hazy since they contained unsedimented particulate iNOS and fragments of iNOS as shown by the multiple bands on the Western blot, FIG. 8; and they appeared much like the particulate fraction of iNOS after it has been resuspended and diluted. The fractions that comprise Peak #2, fractions #17 and 18, were completely clear and contained partially purified soluble iNOS and fragments of iNOS that were not separated from the intact protein by this chromatography procedure as shown by the multiple bands in the Western blot, FIG. 8.

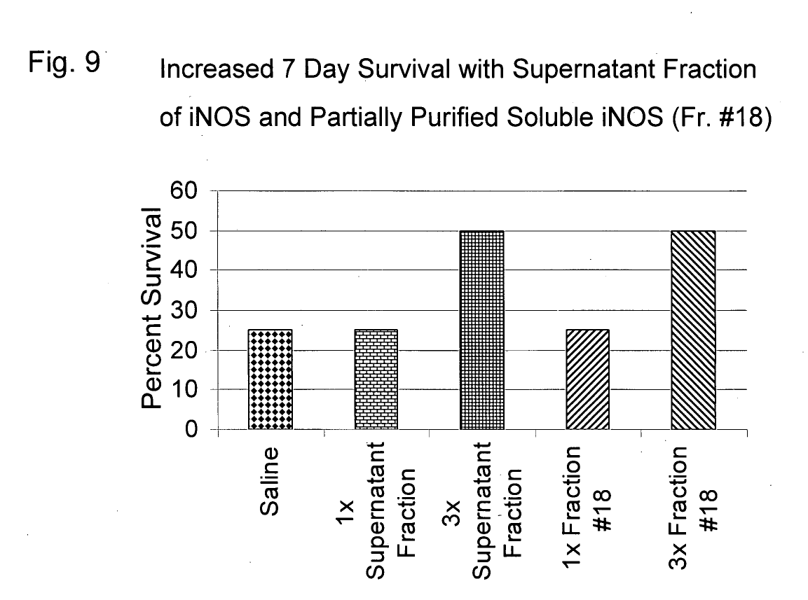

The partially purified soluble iNOS contained in fraction #18 from the G-200 column was tested in our mouse model of sepsis, FIG. 9. Briefly, groups of mice (4 female and 4 male mice per group; N=8) were primed with a sub-lethal dose of LPS; 3.5 hrs later (30 minutes before being challenged with a lethal dose of particulate iNOS), the mice in each group were injected IV with saline, the starting supernatant fraction of iNOS, or Fr. #18 containing the purified soluble iNOS; and 30 minutes later all the mice were challenged with a lethal dose of particulate iNOS. The prior injection of the starting supernatant fraction of iNOS obtained from induced and lysed DLD-1-5B2 cells and the prior injection of Fr. #18 from the Sephadex G-200 column chromatography of the supernatant fraction of iNOS which contained the partially purified soluble iNOS were both found to protect LPS-primed mice from a lethal challenge of particulate iNOS, FIG. 9. Further, the protective effect was found to be dose dependent since at a low dose neither the starting supernatant fraction of iNOS nor fraction #18 which contained the partially purified soluble iNOS were protective. However, at a 3 times higher dose, both the supernatant fraction of iNOS and the partially purified soluble iNOS contained in fraction #18 rescued mice from death by sepsis, FIG. 9. Thus, the supernatant fraction of iNOS from induced and lysed DLD-1-5B2 cells, which was the starting material for the G-200 column chromatography, was protective against a lethal challenge of particulate iNOS. The same protective effect has been shown herein to be contained in fraction #18 following the column chromatography of the supernatant fraction of iNOS. Thus, the partially purified soluble iNOS has been demonstrated to be protective against death by sepsis since more mice survived when administered Fr. #18 as compared to the saline treated group, FIG. 9. Further, these results demonstrated that the partially purified soluble iNOS contained in fraction #18 was able to rescue mice from death by sepsis by antagonizing the deleterious effects exerted by the particulate iNOS. We concluded from these data (1) that soluble intact iNOS or a fragment of iNOS in vivo protects from a lethal challenge of the particulate fraction of iNOS by competing for binding to cellular receptors, (2) that intact soluble iNOS or a fragment of iNOS in vivo is a receptor antagonist for cellular binding by the particulate fraction of iNOS, and (3) that treatment with a receptor antagonist, such as soluble intact iNOS, or a fragment of soluble iNOS that binds to the receptor for particulate iNOS, or an analogue of soluble iNOS that binds to the receptor for particulate iNOS, might be a beneficial therapy for patients with or at risk for developing SIRS, sepsis, severe sepsis and septic shock.

### EXAMPLE X

In order to confirm that the protective effect observed with the partially purified soluble iNOS contained in Peak #2 from the G-200 chromatography was receptor mediated, fraction #12 (Fr12) from Peak #1 and fraction #17 (Fr17) from Peak #2 were dialyzed, concentrated, and radio-iodinated using iodogen and carrier free Na ¹²⁵I. The ¹²⁵I-labeled proteins were then used in a series of experiments similar to those described in Example IX to determine in vivo (1) if cellular binding as measured by organ uptake of the ¹²⁵I—Fr12 and the ¹²⁵I—Fr17 occurs, and (2) if the cellular binding as measured by organ uptake could be inhibited (antagonized) by prior treatment with unlabeled, partially purified soluble iNOS functioning as a receptor antagonist, and thereby, protect the cells (and organs) from the deleterious effects of particulate iNOS. Eight different groups each containing three mice (N=3), Table 1, were primed with a sub-lethal dose of LPS or given a saline injection; 3.5 hrs later they were injected IV with either saline, unlabeled Fr. #12, or unlabeled Fr. #17; 30 min later they were injected IV with 4×10⁶ cpm of either ¹²⁵I—Fr12 (groups #1-4) or ¹²⁵I—Fr17 (groups #5-8); and 60 min later (T=5 hr) they were euthanized by ether anesthesia. Blood samples were obtained from the eye orbital vein while the animals were under deep ether anesthesia immediately prior to death. Prior to dissection, each animal was subjected to transcardial whole-body perfusion with 20 ml of sterile saline to remove blood from all the organs. Skeletal muscle, heart, liver, lungs, spleen, kidneys, intestines, brain and spinal cord were rapidly dissected, weighed, and counted for their content of ¹²⁵I on a scintillation well gamma spectrometer. The results for each mouse as counts per minute (cpm) ¹²⁵I per mg tissue (cpm/mg) was calculated for each organ individually. The average cpm/mg for each organ was then calculated for each group of 3 mice, and the results obtained ranged from a low of 4.8 cpm/mg for brain (saline+saline+¹²⁵I—Fr12 treatment which is group #1) to a high of 695 cpm/mg for spleen (LPS+saline+¹²⁵I—Fr12 treatment which is group #2). The results as average cpm/mg obtained for the heart, spinal cord, intestines and liver with the eight different treatments are summarized in FIG. 10A, FIG. 10B, FIG. 10C, and FIG. 10D, respectively.

In order to compare the changes that resulted from the different treatments in each organ for groups #1-4 which were administered ¹²⁵I—Fr12 and for groups #5-8 which were administered ¹²⁵I—Fr17, the results obtained for the heart, spinal cord, intestines, and liver were normalized (Table 1) to the value found for the saline+saline treated animals for each tracer (group #1 for the ¹²⁵I—Fr12 tracer and group #5 for the ¹²⁵I—Fr17 tracer). Statistically significant differences were calculated by Student's T-test, and P values less than 0.05 were considered significant.

TABLE 1
Normalized CPM/mg as Percentages of
the Saline + Saline Treated Groups

| | T = 0 | | | |
|---|---|---|---|---|
| | saline | LPS | LPS | LPS |
| Tracer @ | T = 3.5 hr | | | |
| 4 hr | saline | saline | Fr12 | Fr17 |
| | Heart | | | |
| ¹²⁵I-Fr12 | 100.0% (G #1) | 186.4% (G #2) | 158.9% (G #3) | 157.9% |
| | | | | (G #4) |
| ¹²⁵I-Fr17 | 100.0% (G #5) | 163.3% (G #6) | 148.7% (G #7) | 124.0% |
| | | | | (G #8) |
| | Spinal Cord | | | |
| ¹²⁵I-Fr12 | 100.0% (G #1) | 187.1% (G #2) | 144.7% (G #3) | 189.7% |
| | | | | (G #4) |
| ¹²⁵I-Fr17 | 100.0% (G #5) | 130.8% (G #6) | 155.7% (G #7) | 107.9% |
| | | | | (G #8) |
| | Intestines | | | |
| ¹²⁵I-Fr12 | 100.0% (G #1) | 201.1% (G #2) | 229.3% (G #3) | 144.8% |
| | | | | (G #4) |
| ¹²⁵I-Fr17 | 100.0% (G #5) | 169.5% (G #6) | 159.3% (G #7) | 105.8% |
| | | | | (G #8) |
| | Liver | | | |
| ¹²⁵I-Fr12 | 100.0% (G #1) | 116.0% (G #2) | 114.9% (G #3) | 121.8% |
| | | | | (G #4) |
| ¹²⁵I-Fr17 | 100.0% (G #5) | 103.5% (G #6) | 122.1% (G #7) | 91.6% |
| | | | | (G #8) |

In Table 1, above as heretofore stated, each group consisted of three adult mice (N=3).

Fraction #12 (Fr12) contained unsedimented particulate iNOS and fragments of iNOS that eluted in the void volume of the Sephadex G-200 column as described in Example IX, and fraction #17 (Fr17) contained intact soluble iNOS and fragments of iNOS, as described in Example IX, that eluted off the G-200 column 25 ml after the unsedimented particulate iNOS contained in fraction 12. The cpm/mg for each of the four treatment groups in each row was normalized to the saline+saline treatment group for that organ and ¹²⁵I-labeled tracer.

In the heart, LPS treatment (group #2) resulted in an 86% increase in the amount of ¹²⁵I—Fr12 (unsedimented particulate iNOS) that was bound and taken-up in the 60 minute period between radio-tracer administration and the animals being euthanized as compared to the saline treated group #1 (P<0.05), and 28% and 29% reductions in ¹²⁵I-uptake were achieved by the prior treatment with the unlabeled, unsedimented particulate iNOS (Fr. #12) to group #3 and with the unlabeled, partially purified soluble iNOS (Fr #17) to group #4, respectively, FIG. 10A and Table 1. In contrast, when ¹²⁵I—Fr17 was tested (groups #5-8), treatment with LPS (group #6) resulted in a 63% increase in radio-label uptake as compared to the saline treated group #5 (P<0.02). Unlabeled Fr. #12 was able to compete for binding and decreased the uptake of ¹²⁵I—Fr17 by 15% in group #7. However, unlabeled partially purified soluble iNOS (Fr. #17) was much more effective at competing for cellular binding and yielded a 39% decrease in radio-label uptake by the heart as demonstrated by group #8, FIG. 10A and Table 1.

In the spinal cord, LPS treatment to group #2 resulted in an 87% increase in the amount of ¹²⁵I—Fr12 (unsedimented particulate iNOS) that was bound and taken-up as compared to the saline treated group #1 (P<0.05), and only unsedimented particulate iNOS (Fr. #12) was able to compete for binding (as shown by the 37% decrease in tissue uptake of ¹²⁵I—Fr12 by group #3). This was evident since no decrease in the amount of radio-labeled tracer uptake was observed with the partially purified soluble iNOS (Fr. #17) treatment in group #4, FIG. 10B and Table 1. When ¹²⁵I —Fr17 was tested (groups #5-8), treatment with LPS in group #6 resulted in a 31% increase in uptake by the spinal cord as compared to the saline treated group #5. This uptake was further increased, (not decreased), by treatment with unsedimented particulate iNOS to group #7 (P<0.05), which may indicate that in the spinal cord particulate iNOS upregulates, activates, or induces its receptor. However, treatment with unlabeled partially purified soluble iNOS (Fr. #17) in group #8 resulted in a 23% decrease in ¹²⁵I—Fr17 uptake by the spinal cord which lowered the LPS induced increase to a mere 8% above that found for the saline+saline treated group of mice (group #5), FIG. 10B and Table 1.

In the intestines, treatment with LPS (group #2) resulted in a more than doubling of the amount of ¹²⁵I—Fr12 (unsedimented particulate iNOS) that was bound and taken-up as compared to the saline treated group #1 (P<0.05). Similar to the spinal cord results, treatment with unlabeled, unsedimented particulate iNOS (Fr. #12) to group #3 showed an additional 28% increase in the amount of radio-labeled particulate iNOS bound and taken-up by the cells in the intestines (P<0.05). The increase observed, following treatment with unlabeled Fr. #12 by the intestines, may again indicate that particulate iNOS can upregulate, activate, or induce its receptor in this organ. However as shown by group #4, administration of partially purified soluble iNOS (unlabeled Fr. #17) successfully competed for binding to the intestinal receptors for particulate iNOS by decreasing the amount of ¹²⁵I—Fr12 bound by the cells. This is demonstrated by the 56% and 84% reduction in uptake of ¹²⁵I—Fr12 as compared to the LPS+saline treated group #2 (P<0.05) and to the LPS+Fr. #12 treated group #3 (P<0.02), respectively, FIG. 10C and Table 1. Thus, soluble iNOS or a fragment of iNOS contained in Fr. #17 effectively bound to the cellular receptors in the intestines, occupied their binding sites, and functioned as a receptor antagonist with respect to the binding of particulate iNOS in the intestines. This is indicated by the decrease in the amount of cellular binding and organ uptake of ¹²⁵I-labeled unsedimented particulate iNOS, illustrated in FIG. 10C and Table 1. When ¹²⁵I —Fr17 was tested in the intestines (groups #5-8), treatment with LPS to group #6 resulted in a 69% increase in uptake of ¹²⁵I—Fr17 by the intestines, as compared to the saline treated group #5 (P <0.02). Unlabeled Fr. #12 (unsedimented particulate iNOS) was a poor receptor antagonist as demonstrated by group #7, since it only reduced by 10% the LPS induced increase in uptake ¹²⁵I—Fr17. In contrast, the unlabeled partially purified soluble iNOS (Fr. #17) administered to group #8 was a very good receptor antagonist since it was successful at competing for cellular receptor binding when given 30 minutes in advance of the tracer. This is evidenced by the 63% and 53% reduction in cellular binding as measured by intestinal uptake of ¹²⁵I—Fr17 when compared to the LPS+saline treated group #6 (P<0.02), and the LPS+unlabeled Fr. #12 treated group #7 (P<0.05), respectively, FIG. 10C and Table 1. Thus, the partially purified soluble iNOS (Fr. #17) reduced the LPS induced increase in cellular binding in group #8 to only 6% above that found for the saline+saline treated group of mice (group #5). Further, soluble iNOS or a fragment of iNOS contained in Fr. #17, when administered 30 minutes in advance of the tracer, effectively antagonized the binding of ¹²⁵I—Fr17 to the cellular receptors in the intestines as demonstrated by the comparing of results obtained for group #8 to those of group #6, FIG. 10C and Table 1. Therefore, in the intestines the intact soluble iNOS, or a fragment of iNOS contained in fraction #17 from the G-200 column chromatography, functioned as a receptor antagonist for binding by the ¹²⁵I—Fr12. This is shown by the 56% reduction in uptake observed when comparing group #2 and group #4, FIG. 10C and Table 1, and of the binding by the ¹²⁵I—Fr17 to the cellular receptors which had a 64% reduction in uptaked observed when comparing group #6 and group #8, FIG. 10C and Table 1.

In the liver, no discernible changes were observed in the uptake of ¹²⁵I—Fr12 (groups #1-4) or of ¹²⁵I—Fr17 (groups #5-8) with LPS treatment or with LPS treatment followed by competition with unlabeled Fr. #12 and unlabeled Fr. #17, as compared to the saline+saline treated groups of mice (groups #1 and 5), FIG. 10D and Table 1. The liver contained the highest total amount of ¹²⁵I due mainly to its size. When compared to other organs on a cpm/mg basis, it was found that the content of radio-tracer in the liver was similar to many of the other organs examined.

In summary, treatment with LPS prior to the administration of the ¹²⁵I-labeled tracers resulted in an increase in organ uptake of the ¹²⁵I-labeled tracer through cellular binding to receptors in the heart, spinal cord, and intestines. However, no such increase was observed in the liver. If treated with unlabeled, partially purified soluble iNOS 30 minutes prior to the administration of the ¹²⁵I-labeled tracer, the cellular receptors were at least partially blocked as demonstrated by the ability of the unlabeled, soluble iNOS or a fragment of iNOS contained in fraction #17, to compete for cellular binding, and thereby decrease the amount of ¹²⁵I-labeled tracer found in the heart, spinal cord, and intestines. Thus, the unlabeled, soluble iNOS or a fragment of iNOS contained in fraction #17 was concluded to be functioning as a receptor antagonist with respect to the binding of ¹²⁵I-labeled unsedimented particulate iNOS contained in fraction #12, and with respect to the binding of ¹²⁵I-labeled partially purified soluble iNOS contained in fraction #17.

Since particulate iNOS has been shown herein to be centrally involved in the pathology of sepsis, since intact soluble iNOS or a fragment of iNOS has been shown herein to rescue mice from death by sepsis, and since intact soluble iNOS or a fragment of iNOS has herein been demonstrated to be a receptor antagonist in the heart, spinal cord and intestines, patients with or at risk for developing SIRS, sepsis, severe sepsis, and septic shock with a particulate iNOS, may be treated with a receptor antagonist. Such treatment is indicated to be a beneficial therapy for these life threatening conditions. The receptor antagonist could be intact soluble iNOS, or a fragment of soluble iNOS that binds to the receptor and competes for binding with particulate iNOS, or an analogue of soluble iNOS that binds to the receptor and competes for binding with particulate iNOS.

While in the foregoing, embodiments representing the carrying out of the present invention have been set forth in considerable detail for the purposes of making a complete disclosure of the invention, it may be apparent to those of skill in the art that numerous changes may be made in such detail without departing from the spirit and principles of the invention.

What is claimed is:

1. A therapeutic agent for the treatment of an illness in a mammalian subject in which inducible nitric oxide synthase (iNOS) is present, comprising:
a receptor antagonist that inhibits the cellular binding of particulate inducible nitric oxide synthase (iNOS).

2. The therapeutic agent of claim 1 in which said receptor antagonist comprises a soluble fraction of inducible nitric oxide synthase (iNOS).

3. The therapeutic agent of claim 2 in which said soluble fraction of inducible nitric oxide synthase (iNOS) comprises a soluble fraction of inducible nitric oxide synthase (iNOS) eluded from a chromatography column.

4. The therapeutic agent of claim 3 in which said soluble fraction of inducible nitric oxide synthase (iNOS) eluded from a chromatographic column comprises inducible nitric oxide synthase from induced cells.

5. The therapeutic agent of claim 1 in which said receptor antagonist comprises intact soluble iNOS.

6. The therapeutic agent of claim 1 in which said receptor antagonist comprises a fragment of soluble iNOS.

7. The therapeutic agent of claim 1 in which said receptor antagonist comprises an analogue of soluble iNOS.

8. The therapeutic agent of claim 1 in which said illness is selected from the group consisting essentially of:
systemic inflammatory response syndrome (SIRS), sepsis, severe sepsis, and septic shock syndrome.

9. A method for the treatment of illness in a mammalian subject in which inducible nitric oxide synthase is present, comprising the step of:
introducing into the mammalian subject, a receptor antagonist that inhibits the cellular binding of particulate inducible nitric oxide synthase (iNOS).

10. The method of claim 9 in which said receptor antagonist comprises a soluble fraction of inducible nitric oxide synthase (iNOS).

11. The method of claim 10 in which said soluble fraction of inducible nitric oxide synthase (iNOS) comprises a soluble fraction of inducible nitric oxide synthase (iNOS) eluded from a chromatography column.

12. The method of claim 11 in which said soluble fraction of inducible nitric oxide synthase (iNOS) eluded from a chromatographic column comprises inducible nitric oxide synthase from induced cells.

13. The method of claim 9 in which said receptor antagonist comprises intact soluble iNOS.

14. The method of claim 9 in which said receptor antagonist comprises a fragment of soluble iNOS.

15. The method of claim 9 in which said receptor antagonist comprises an analogue of soluble iNOS.

16. The method of claim 9 in which said illness is selected from the group consisting essentially of:
systemic inflammatory response syndrome (SIRS), sepsis, severe sepsis, and septic shock syndrome.
